(19) United States
(12) Patent Application Publication
(10) Pub. No.: US 2005/0182489 A1
(43) Pub. Date: Aug. 18, 2005

(54) Intraocular lens adapted for adjustment via laser after implantation

(76) Inventor: Gholam A. Peyman, New Orleans, LA (US)

Correspondence Address: Bell, Boyd & Lloyd LLC, P.O. Box 1135, Chicago, IL, 60690-1135, US

(21) Appl. No.: 11/106,922

(22) Filed: Apr. 15, 2005

Related U.S. Application Data
(63) Continuation-in-part of application No. 10/958,826, filed on Oct. 4, 2004.
Application No. 10/958,826 is a continuation-in-part of application No. 10/272,402, filed on Oct. 17, 2002.
Application No. 10/958,826 is a continuation-in-part of application No. 10/784,169, filed on Feb. 24, 2004.
Application No. 10/784,169 is a continuation-in-part of application No. 10/406,558, filed on Apr. 4, 2003.
Application No. 10/784,169 is a continuation-in-part of application No. 10/356,730, filed on Feb. 3, 2003.
Application No. 10/356,730 is a continuation-in-part of application No. 09/843,141, filed on Apr. 27, 2001, now Pat. No. 6,551,307.
Application No. 10/356,730 is a continuation-in-part of application No. 09/986,141, filed on Nov. 7, 2001.
(60) Provisional application No. 60/449,617, filed on Feb. 26, 2003.

(51) Int. Cl. A61F 2/16; G02C 7/04; B29D 11/00; A61B 18/20

(52) U.S. Cl. 623/6.22; 351/177; 606/5; 623/6.31; 264/1.37

(57) ABSTRACT

A method and apparatus is provided for an intraocular device adapted for adjustment via a laser after implantation into an eye. The intraocular device is inserted into an eye, and one or more optical characteristics of the eye, including the intraocular device, are measured. Then, a groove configuration is determined for the intraocular device, and the configuration is ablated into the intraocular device with a short pulse laser.

### CROSS REFERENCE TO RELATED APPLICATIONS

This patent application is a continuation-in-part of U.S. Non-Provisional application Ser. No. 10/958,826, filed on Oct. 4, 2004, which is a continuation-in-part of U.S. Non-Provisional application Ser. No. 10/272,402, filed on Oct. 17, 2002; and is a continuation-in-part of U.S. Non-Provisional application Ser. No. 10/784,169 filed on Feb. 24, 2004, which is a continuation-in-part of U.S. Non-Provisional application Ser. No. 10/406,558, filed on Apr. 4, 2003 which claims the benefit of U.S. Provisional Application No. 60/449,617, filed on Feb. 26, 2003, and is a continuation-in-part of U.S. Non-Provisional application Ser. No. 10/356,730, filed on Feb. 3, 2002, which is a continuation-in-part of U.S. Non-Provisional application Ser. No. 09/843,141, filed on Apr. 27, 2001; and is a continuation-in-part of U.S. Non-Provisional application Ser. No. 09/986,141, filed on Nov. 7, 2001, the entire contents of each of which are incorporated herein by reference.

### FIELD OF THE INVENTION

The present invention relates to an intraocular implant adapted for adjustment after implantation into the human eye. More specifically, the present invention relates to intraocular implants or lenses adapted for adjustment via a short pulse laser (e.g., a femtosecond, picosecond or attosecond laser). The short pulse laser ablates away a portion of the intraocular lens while in situ; thus, forming grooves that generate diffractive effects.

### BACKGROUND OF THE INVENTION

There are many ocular diseases for which a patient's vision can be improved by positioning optical implants in the eye; however, post-fabrication adjustment of these implants is often difficult. Of course, such adjustments of the lenses' optical characteristics is beneficial in various ophthalmic lens types. For example, cataract patients would benefit from post-implant power adjustability of an IOL implant. In another case, posterior chamber phakic IOLs could benefit from post-implant power adjustability since biometry cannot insure proper power selection. Additionally, contact lenses would benefit from post-fabrication adjustment to limit the number of lenses that needed to be maintained in inventories or to more exactly match a particular lens to a specific eye's needs.

The correction of cataracts would also benefit from lenses that could be adjusted post-fabrication. Cataracts are major cause of blindness in the world and the most prevalent ocular disease. Visual disability from cataracts accounts for more than 8 million physician office visits per year. When the disability from cataracts affects or alters an individual's activities of daily living, surgical lens removal with intraocular lens implantation is the preferred method of treating the functional limitations. In the United States, about 2.5 million cataract surgical procedures are performed annually, making it the most common surgery for Americans over the age of 65. About 97 percent of cataract surgery patients receive intraocular lens implants, with the annual costs for cataract surgery and associated care in the United States being upwards of $4 billion.

A cataract is any opacity of a patient's lens, whether it is a localized opacity or a diffuse general loss of transparency. To be clinically significant, however, the cataract must cause a significant reduction in visual acuity or a functional impairment. A cataract occurs as a result of aging or hereditary factors, trauma, inflammation, metabolic or nutritional disorders, or radiation. Age-related cataract conditions are the most common.

In treating a cataract, the surgeon removes material from the lens capsule and replaces it with an intraocular lens (IOL) implant. The typical IOL provides a selected focal length that allows the patient to have fairly good distance vision. Since the lens can no longer accommodate, the patient typically needs prescription eyeglasses for reading.

The surgeon selects the power of the IOL based on analysis of biometry of the patient's eye prior to the surgery. In a significant number or cases, after the patient's eye has healed from the cataract surgery, there is a refractive error beyond the margin of error in the biometric systems. Thus, there remain intractable problems in calculating the proper power of an IOL for any particular patient. To solve any unpredicted refractive errors following IOL implantation, the ophthalmologist can perform a repeat surgery to replace the IOL, or the patient can live with the refractive error and may require prescription eyeglasses to correct for both near and distant vision. However, even repeated surgeries can be ineffective in correcting the problem.

What is needed is a lens system that provides means for post-fabrication or post-implant adjustment of optical characteristics and dioptic power. What also is needed is a lens system that can correct higher order aberrations.

### SUMMARY OF THE INVENTION

In one embodiment of the present invention, an intraocular device is adjusted via a laser after implantation into an eye. The intraocular device is inserted into an eye, and one or more optical characteristics of the eye, including the intraocular device, are measured. Then, a groove configuration is determined for the intraocular device, and the configuration is ablated into the intraocular device with a short pulse laser. In another embodiment, the eye is allowed to heal after inserting said intraocular device before the optical characteristics of the eye are measured.

In one embodiment, the short pulse laser is a picosecond laser, a femtosecond laser or an attosecond laser. In another embodiment, the intraocular device is a foldable lens or a hard lens. In still another embodiment, the intraocular device includes a polymer, silicone or acrylic.

In one embodiment, a contact lens is adjusted via a laser when placed on an eye. The contact lens is placed on the eye and the optical characteristics of the eye and lens are measured. Then, a groove configuration is determined for the contact lens, and the configuration is ablated into the lens with a short pulse laser. In one embodiment, the short pulse laser is a picosecond laser, a femtosecond laser or an attosecond laser. In another embodiment, the contact lens is a soft lens or a gas permeable lens. In still another embodiment, the contact lens includes a polymer, silicone or acrylic.

In one embodiment, an optical system is adapted by determining a groove configuration for a portion of the optical system and ablating with a short pulse laser the groove configuration into the portion of the optical system. The groove configuration causes a diffraction effect that improves the optical performance of the optical system. In various embodiments, the portion of the optical system is an intraocular device, an intraocular lens, a natural lens, a contact lens, an eyeglass lens or a corrective lens.

In one embodiment, a computer program product is provided, including a computer usable medium having computer readable program code embodied therein configured to calculate a groove configuration. The computer program product causes a computer to determine one or more optical characteristics of an optical system and causes a computer to determine a groove configuration. The groove configuration causes a diffraction effect that improves the optical performance of the optical system.

In another embodiment, the computer program product also causes a computer to ablate said groove configuration into a portion of the optical system using a short pulse laser. In various embodiments, the portion is an intraocular device, an intraocular lens, a natural lens, a contact lens, an eyeglass lens or a corrective lens.

Additional features and advantages of the present invention are described in, and will be apparent from, the following Detailed Description of the Invention and the figures.

### BRIEF DESCRIPTION OF THE FIGURES

FIG. 1 illustrates a cross section of an eye with an intraocular lens adapted for adjustment via laser after implantation, in accordance with one embodiment of the present invention.

FIG. 2 illustrates a cross section of the intraocular lens of FIG. 1.

FIG. 3 illustrates a frontal view of the intraocular lens of FIGS. 1 and 2.

FIG. 4 illustrates the process of implanting an intraocular lens capable of being adjusted via laser after implantation into the eye in accordance with one embodiment of the present invention.

FIG. 5 illustrates a cross section of an eye with a contact lens adapted for adjustment via laser on the eye, in accordance with one embodiment of the present invention.

FIG. 6 illustrates a cross section of the contact lens of FIG. 5.

FIG. 7 illustrates a frontal view of the contact lens of FIGS. 5 and 6.

FIG. 8 illustrates the process of determining a groove configuration in accordance with one embodiment of the present invention.

FIG. 9 illustrates a schematic of a general purpose computer upon which the process of FIG. 8 can be embodied in program code in accordance with one embodiment of the present invention.

### DETAILED DESCRIPTION OF THE INVENTION

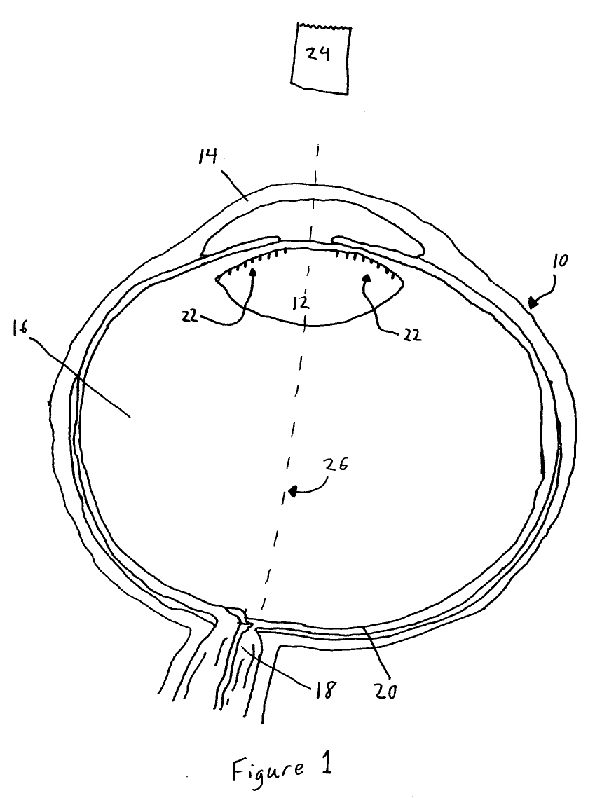

The optical system of FIG. 1 is an eye 10 in which the lens has been replaced by an IOL 12. The eye 10 generally consists of a cornea 14, the IOL 12, vitreous 16, the optic nerve 18 and a retina 20. IOL 12 is preferably foldable, but may be hard or any other suitable type. Further, the IOL 12 is preferably made from a polymer; however, the IOL 12 can be silicone, acrylic or any other suitable material.

FIG. 1 shows an optical system which was modified by ablating grooves 22 into a portion of the optical system using a short pulse laser 24. The grooves 22 produce a diffractive effect when light passes through the optical system, improving the optical system's performance. Preferably, the grooves 22 are ablated into an IOL 12; however, the grooves can be ablated into a contact lens, eye glasses, the natural lens of the eye 10 or any other suitable portion of the optical system. The grooves 22 are preferably about 1 nanometer to about 50 microns deep and about 1 nanometer to about 50 microns wide and are spaced about 1 nanometer to about 50 microns apart from each other; however the grooves 22 can have any suitable depth, width and/or spacing.

Preferably, the IOL 12 is placed in situ by a procedure in which an incision is made in the eye 10, the original lens is removed, the IOL 12 is positioned within the eye 10, and the incision is closed; however, any suitable procedure, including procedures in which the original lens or a portion of the original lens is not removed, may be used. The IOL 12 can be used in conjunction with existing contacts, glasses, the natural lens, another IOL or any other suitable optical device, or the IOL 12 can be used alone. Further, the IOL 12 can be positioned in any suitable chamber (e.g., anterior or posterior) or within any suitable tissue or structure. The IOL 12 also can be attached to the existing or natural lens in any suitable manner, or the IOL 12 can be detached from or replace the existing or natural lens.

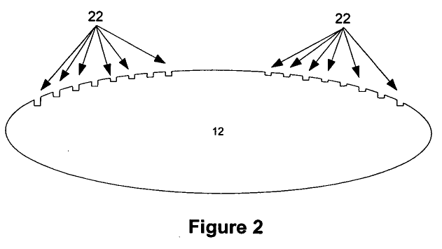

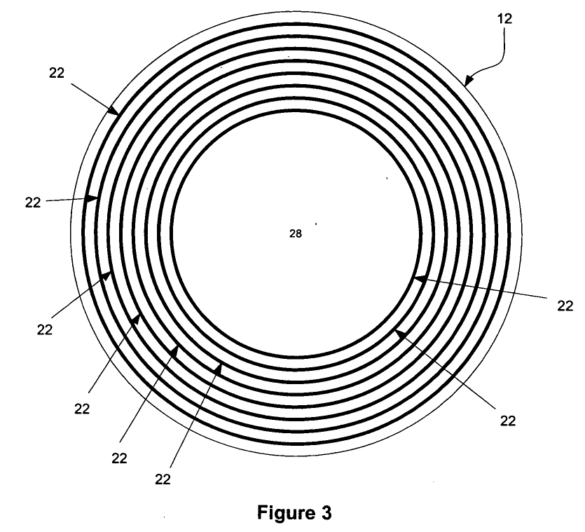

One reason lenses or devices having grooves 22 are advantageous over non-diffractive lenses is that the grooves 22 can be created in situ after the eye has healed from implantation of any IOL 12 or any other procedure. Thus, the already completed healing process will not change the optical characteristics of the eye after the grooves 22 are created and the patient will enjoy better vision as a result. After the IOL 12 is placed in situ, it is modified to more precisely correct any remaining refractive error in the eye or facilitate restoration of the far vision in the eye to precisely match the particular characteristics of the eye 10 by ablating a portion of the IOL 12 using a short pulse laser 24. Preferably, the short pulse laser is a picosecond laser; however, the laser can be a femtosecond laser, an attosecond laser or any other suitable short pulse laser or any other suitable laser. As illustrated in FIGS. 2 and 3, the laser forms grooves 22 in the IOL 12. The grooves are preferably substantially circular grooves that are formed concentrically about the main optical axis 26. As shown specifically in FIGS. 1-3, grooves 22 are spaced approximately equidistant apart from each other and form gradually progressive circles that begin at or about at the center portion of the IOL 12 and extend to or adjacent to the peripheral portion of the IOL 12. However, the grooves 22 can be any suitable configuration, distance apart and/or position on the IOL 12 desired. Further, the grooves 22 can be regularly or irregularly spaced, non-concentric, configured as line/curve segments or any other suitable path rather than as closed loops and/or discontinuous. The grooves 22 can also overlap and/or vary in width, depth, and/or shape.

Center portion 28 is preferably left unaltered such that light passing therethrough does not impinge or is not altered or diffracted by any grooves. However, if desired, grooves can be positioned on center portion 28. With the center portion 28 unaltered, the IOL 12 can exhibit multifocal properties. That is, the center portion 28 can be adjusted to correct for far vision and the peripheral portion can correct for close distance, such as for reading. Although, the center portion 28 and/or the peripheral portion can be configured to correct for any type of vision.

The edges formed by the ablation are preferably smooth, so the application of a resin is not necessary to smooth over rough portions; however, if desired, a resin can be used to smooth the surfaces of any portion of the IOL 12 or any other suitable purpose. Preferably, the grooves 22 have valleys so small that only a short pulse laser could form them; however, larger valleys may be formed as needed depending on the particular characteristics of the eye 10.

As light passes though the IOL 12, the grooves 22 cause diffractive effects and/or prismatic effects, bending the light in a predictable manner. Preferably, the grooves 22 are arranged such that their diffractive effects cause light entering the eye 10 to converge at a more ideal focal point within the eye, thus correcting any myopia or hyperopia of the eye 10. It should be noted that the grooves 22 of FIGS. 1 through 3 are for illustrative purposes only, and that proper groove configuration and number of grooves can depend upon the characteristics of the eye 10, including the position and configuration of the cornea 14, the IOL 12, the retina 20 and any other possible exterior or interior factors.

Suitable configuration of the grooves 22 preferably results in the IOL 12 having multiple focal points; however, the lens can have one focal point or any number of focal points desired. For example, differing peripheral areas can have different refractive and/or diffractive properties. That is, a radial portion adjacenty the periphery of the IOL 12 can be configured to correct far vision, while a median radial area can be configured for close or reading vision. As a result of multifocality, the IOL 12 can bring both near and far objects into focus, reducing or eliminating the need for corrective lenses for reading or other activities. Further, because different wavelengths of light diffract at different angles, the IOL 12 can selectively focus different colors of light at different focal lengths.

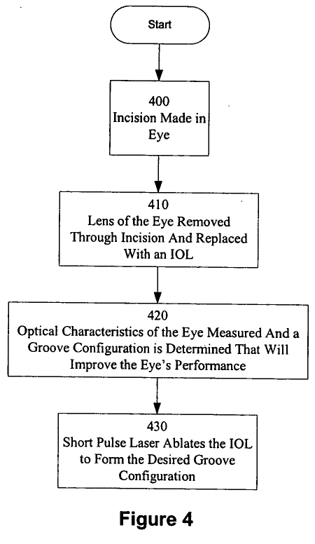

FIG. 4 illustrates the preferred process of adapting an intraocular device (e.g., IOL 12) via a laser after implantation; however other suitable processes may be used. At step 400, an incision is made in the eye. Then, at step 410, the lens of the eye is removed through the incision and replaced with an IOL. Preferably, the eye is allowed to heal before further steps are taken; however, the process can continue as part of the same operation that implants the IOL or in any other suitable manner. At step 420, the optical characteristics of the eye are measured and a groove configuration is determined that will improve the eye's performance. Preferably, the optical characteristics of the eye are measured, or mapped, by directing light into the eye and noting the behavior of the light returning from the back of the eye; however, any suitable method of measuring the optical characteristics of the eye may be used. Then, at step 430, a short pulse laser ablates the IOL to form the desired groove configuration. By allowing the eye to heal from implanting the IOL before measuring the eye's optical characteristics, it is less likely the characteristics will change significantly after the groove configuration is ablated into the IOL.

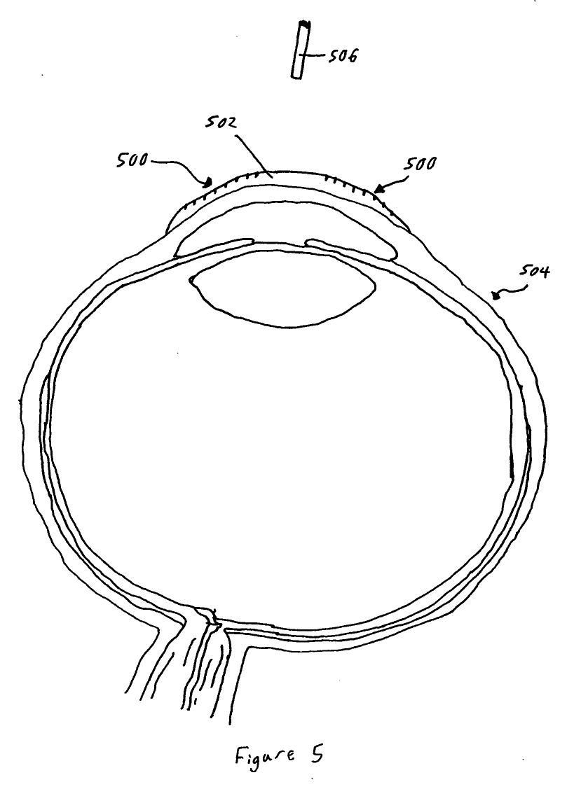

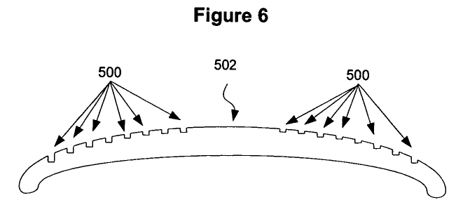

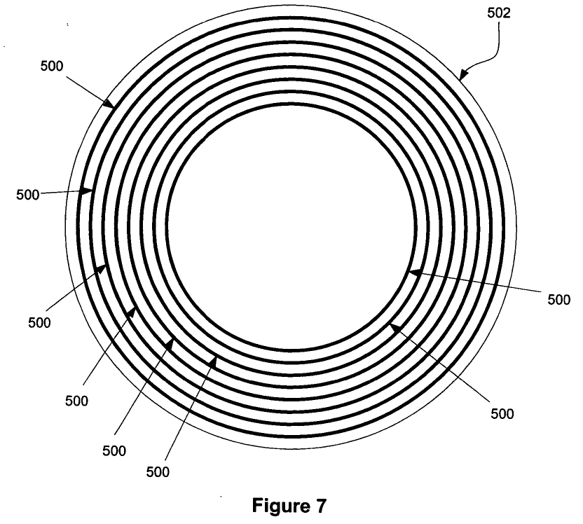

As illustrated by FIGS. 5 through 7, grooves 500 can also be ablated into a contact lens 502 placed on the eye 504. It should be noted that eye glasses or other optical devices with similar grooves ablated into them could be used in addition to or instead of contact lens 502. The lens 502 is preferably made from a polymer; however, the lens 502 can be silicone, acrylic or any other suitable material. The lens 502 can also be soft, gas permeable or any other suitable type. Further, the lens 502 can be used in conjunction with an IOL or other optical device. Once the lens 502 is in place, the combined optical characteristics of the lens 502 and eye 504 combination are measured and a groove configuration is determined that will improve the optical performance of the lens 502 and the eye 504. Preferably, the optical characteristics of the contact lens and eye are measured, or mapped, by directing laser light through the contact lens and into the eye and noting the behavior of the light returning from the back of the eye; however, any suitable method of measuring the optical characteristics of the contact lens and eye may be used. Then, a short pulse laser 506 or any other suitable laser ablates the lens 502 to form the desired groove configuration.

As light passes through the lens 502, the grooves produce diffractive effects, bending the light in a predictable manner. Preferably, the grooves 500 are arranged such that their diffractive effects cause light entering the eye 504 to converge at a more ideal focal point within the eye 504 than the focal point produced by the lens 502 and eye 504 before the ablation. It should be noted that the grooves 500 of FIGS. 5 through 7 are for illustrative purposes only, and that proper groove configuration and number of grooves will depend upon the characteristics of the eye 504 and the lens 502.

Preferably, the groove pattern to be ablated into a portion of an optical system is determined after measuring the optical system's characteristics, including the portion to be ablated; however, the groove pattern can be determined without measuring the portion to be ablated. For example, if a contact lens or eye glasses are to be ablated, the behavior of the lens or glasses can be known without measurement (e.g., a particular contact lens is known to have been manufactured to be a −1.25 diopter lens). Thus, once measurements of the eye are made to determine which type of contact lens or eye glasses to use, the groove pattern can be determined without further measurement; instead using the known or assumed lens characteristics. For example, the ideal contact lens for a particular eye may be determined after measurements of the eye to be a −1.264 diopter lens. A groove configuration can then be determined that will change a −1.25 diopter lens into a −1.264 diopter lens without the need to measure the −1.25 diopter lens. This illustrates another advantage of using lenses or devices with diffraction-causing grooves (e.g., grooves 22) rather than traditional non-diffractive lenses: a doctor can, without the need to special order, provide patients with a greater variety of lens powers than the doctor actually stores in the office.

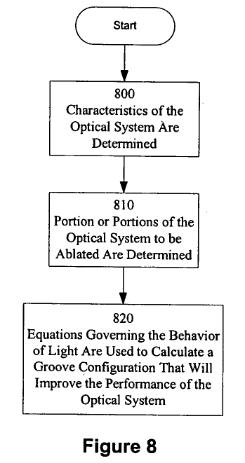

The groove configuration is preferably calculated using a computer; however, the configuration can be generated using any other suitable means. FIG. 8 shows the preferred process of determining a groove configuration; however, any other suitable process can be used. At step 800, the characteristics of the optical system are determined. The characteristics can be determined by measurement and/or any other method. At step 810, the portion or portions of the optical system to be ablated are determined. Then, at step 820, equations governing the behavior of light (e.g., diffraction and refraction equations) well known in the art are used to calculate a groove configuration that will improve the performance of the optical system.

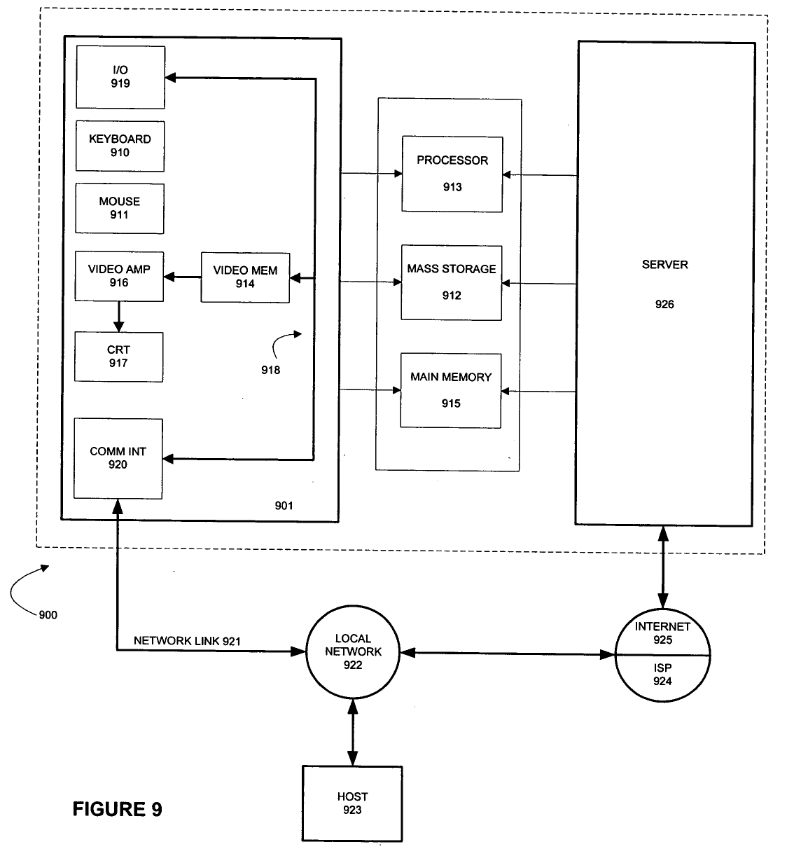

The groove configuration calculation process of FIG. 8 can be implemented as computer software in the form of computer readable program code executed in a general purpose computing environment such as environment 900 illustrated in FIG. 9. A keyboard 910 and mouse 911 are coupled to a system bus 918. The keyboard and mouse are for introducing user input to the computer system and communicating that user input to central processing unit (CPU) 913. Other suitable input devices may be used in addition to, or in place of, the mouse 911 and keyboard 910. I/O (input/output) unit 919 coupled to bi-directional system bus 918 represents such I/O elements as a printer, A/V (audio/video) I/O, etc.

Computer 901 may include a communication interface 920 coupled to bus 918. Communication interface 920 provides a two-way data communication coupling via a network link 921 to a local network 922. For example, if communication interface 920 is an integrated services digital network (ISDN) card or a modem, communication interface 920 provides a data communication connection to the corresponding type of telephone line, which comprises part of network link 921. If communication interface 920 is a local area network (LAN) card, communication interface 920 provides a data communication connection via network link 921 to a compatible LAN. Wireless links are also possible. In any such implementation, communication interface 920 sends and receives electrical, electromagnetic or optical signals which carry digital data streams representing various types of information.

Network link 921 typically provides data communication through one or more networks to other data devices. For example, network link 921 may provide a connection through local network 922 to local server computer 923 or to data equipment operated by ISP 924. ISP 924 in turn provides data communication services through the world wide packet data communication network now commonly referred to as the “Internet” 925. Local network 922 and Internet 925 both use electrical, electromagnetic or optical signals which carry digital data streams. The signals through the various networks and the signals on network link 921 and through communication interface 920, which carry the digital data to and from computer 901, are exemplary forms of carrier waves transporting the information.

Processor 913 may reside wholly on client computer 901 or wholly on server 926 or processor 913 may have its computational power distributed between computer 901 and server 926. Server 926 symbolically is represented in FIG. 9 as one unit, but server 926 can also be distributed between multiple “tiers”. In one embodiment, server 926 comprises a middle and back tier where application logic executes in the middle tier and persistent data is obtained in the back tier. In the case where processor 913 resides wholly on server 926, the results of the computations performed by processor 913 are transmitted to computer 901 via Internet 925, Internet Service Provider (ISP) 924, local network 922 and communication interface 920. In this way, computer 901 is able to display the results of the computation to a user in the form of output.

Computer 901 includes a video memory 914, main memory 915 and mass storage 912, all coupled to bi-directional system bus 918 along with keyboard 910, mouse 911 and processor 913. As with processor 913, in various computing environments, main memory 915 and mass storage 912, can reside wholly on server 926 or computer 901, or they may be distributed between the two.

The mass storage 912 may include both fixed and removable media, such as magnetic, optical or magnetic optical storage systems or any other available mass storage technology. Bus 918 may contain, for example, thirty-two address lines for addressing video memory 914 or main memory 915. The system bus 918 also includes, for example, a 32-bit data bus for transferring data between and among the components, such as processor 913, main memory 915, video memory 914 and mass storage 912. Alternatively, multiplex data/address lines may be used instead of separate data and address lines.

In one embodiment of the invention, the microprocessor is manufactured by Intel, such as the 80X86 or Pentium-type processor. However, any other suitable microprocessor or microcomputer may be utilized. Main memory 915 is comprised of dynamic random access memory (DRAM). Video memory 914 is a dual-ported video random access memory. One port of the video memory 914 is coupled to video amplifier 916. The video amplifier 916 is used to drive the cathode ray tube (CRT) raster monitor 917. Video amplifier 916 is well known in the art and may be implemented by any suitable apparatus. This circuitry converts pixel data stored in video memory 914 to a raster signal suitable for use by monitor 917. Monitor 917 is a type of monitor suitable for displaying graphic images.

Computer 901 can send messages and receive data, including program code, through the network(s), network link 921, and communication interface 920. In the Internet example, remote server computer 926 might transmit a requested code for an application program through Internet 925, ISP 924, local network 922 and communication interface 920. The received code may be executed by processor 913 as it is received, and/or stored in mass storage 912, or other non-volatile storage for later execution. In this manner, computer 901 may obtain application code in the form of a carrier wave. Alternatively, remote server computer 926 may execute applications using processor 913, and utilize mass storage 912, and/or video memory 915. The results of the execution at server 926 are then transmitted through Internet 925, ISP 924, local network 922 and communication interface 920. In this example, computer 901 performs only input and output functions.

Application code may be embodied in any form of computer program product. A computer program product comprises a medium configured to store or transport computer readable code, or in which computer readable code may be embedded. Some examples of computer program products are CD-ROM disks, ROM cards, floppy disks, magnetic tapes, computer hard drives, servers on a network, and carrier waves.

The computer systems described above are for purposes of example only. The groove configuration calculation process of FIG. 8 can be implemented in any type of computer system or programming or processing environment.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A method of altering the optical characteristics of an intraocular device, said method comprising:
inserting said intraocular device into an eye;
measuring one or more optical characteristics of said eye, wherein said eye includes said intraocular device;
determining a groove configuration for said intraocular device; and
ablating with a short pulse laser said groove configuration into said intraocular device.

2. The method of claim 1, further comprising:
waiting for said eye to heal after inserting said intraocular device into said eye before measuring said one or more optical characteristics.

3. The method of claim 1, wherein said short pulse laser is selected from the group consisting of a picosecond laser, a femtosecond laser and an attosecond laser.

4. The method of claim 1, wherein said intraocular device is selected from the group consisting of a foldable lens and a hard lens.

5. The method of claim 1, wherein said intraocular device includes a material selected from the group consisting of a polymer, silicone and acrylic.

6. A method of altering the optical characteristics of a contact lens, said method comprising:
placing said contact lens on an eye;
measuring one or more optical characteristics of said eye and said contact lens;
determining a groove configuration for said contact lens; and
ablating with a short pulse laser said groove configuration into said contact lens.

7. The method of claim 6, wherein said short pulse laser is selected from the group consisting of a picosecond laser, a femtosecond laser and an attosecond laser.

8. The method of claim 6, wherein said contact lens is selected from the group consisting of a soft lens and a gas permeable lens.

9. The method of claim 6, wherein said contact lens includes a material selected from the group consisting of a polymer, silicone and acrylic.

10. A method of changing the optical properties of an optical system, said method comprising:
determining a groove configuration for a portion of said optical system, wherein said groove configuration causes a diffractive effect that improves the optical performance of said optical system; and
ablating with a short pulse laser said groove configuration into said portion.

11. The method of claim 11, wherein said portion is selected from the group consisting of an intraocular device, an intraocular lens, a natural lens, a contact lens, an eyeglass lens and a corrective lens.

12. A system for correcting the refractive error in an eye comprising:
an intraocular device suitable for insertion into the eye;
a short pulse laser; and
one or more grooves, wherein said one or more grooves are ablated into said intraocular device by said short pulse laser, and wherein said one or more grooves are configured to improve one or more optical characteristics of said eye.

13. The system of claim 12, wherein said short pulse laser is selected from the group consisting of a picosecond laser, a femtosecond laser and an attosecond laser.

14. The system of claim 12, wherein said intraocular device is selected from the group consisting of a foldable lens and a hard lens.

15. The system of claim 12, wherein said one or more grooves are positioned on said intraocular device such that at least some light passing through said pupil is diffracted by said one or more grooves.

16. The system of claim 12, wherein said grooves form substantially concentric circles.

17. The system of claim 12, wherein said intraocular device includes a material selected from the group consisting of a polymer, silicone and acrylic.

18. A system for correcting vision in an eye comprising:
a contact lens suitable for placement onto the eye;
a short pulse laser; and
one or more grooves, wherein said one or more grooves are ablated into said contact lens by said short pulse laser, and wherein said one or more grooves are configured to improve one or more optical characteristics of said eye and said contact lens.

19. The system of claim 18, wherein said short pulse laser is selected from the group consisting of a picosecond laser, a femtosecond laser and an attosecond laser.

20. The system of claim 18, wherein said contact lens is selected from the group consisting of a soft lens and a gas permeable lens.

21. The system of claim 18, wherein said contact lens includes a material selected from the group consisting of a polymer, silicone and acrylic.

22. An optical system adaptation device comprising:
a short pulse laser; and
one or more grooves, wherein said one or more grooves are ablated into a portion of an optical system by said short pulse laser, and wherein said one or more grooves are configured to improve one or more optical characteristics of said optical system.

23. The optical system adaptation device of claim 22, wherein said portion is selected from the groups consisting of an intraocular device, an intraocular lens, a natural lens, a contact lens, an eyeglass lens and a monocle.

24. A computer program product comprising:
a computer usable medium having computer readable program code embodied therein configured to calculate a groove configuration, said computer program product comprising:
computer readable code configured to cause a computer to determine one or more optical characteristics of an optical system; and
computer readable code configured to cause a computer to determine said groove configuration, wherein said groove configuration causes a diffraction effect that improves the optical performance of said optical system.

25. The computer program product of claim 24, further comprising:
computer readable code configured to cause a computer to ablate said groove configuration into a portion of said optical system using a short pulse laser.

26. The computer program product of claim 24, wherein said portion is selected from the groups consisting of an intraocular device, an intraocular lens, a natural lens, a contact lens, an eyeglass lens and a monocle.
